(19) United States
(12) Patent Application Publication
(10) Pub. No.: US 2023/0310756 A1
(43) Pub. Date: Oct. 5, 2023

(54) INJECTION DEVICE

(71) Applicant: Simon Williams Pharma Consulting LLC, Gibbsboro, NJ (US)

(72) Inventors: Simon Hamel, Knowlton (CA); Sylvain Coultier, Magog (CA)

(73) Assignee: Simon Williams Pharma Consulting LLC, Gibbsboro, NJ (US)

(21) Appl. No.: 17/803,031

(22) PCT Filed: Jul. 23, 2020
(86) PCT No.: PCT/CA2020/000093

§ 371 Date: Jan. 18, 2022

(30) Foreign Application Priority Data
Jul. 24, 2019 (CA) 3,050,501

(51) Int. Cl. A61M 5/32; A61M 5/315

(52) U.S. Cl. CPC A61M 5/3234; A61M 5/31511

(57) ABSTRACT

An injection device (10) comprising a housing (12), a needle hub (38) slidable within the housing (12), a needle (40) having a distal end piercing tip (42) and a proximal end piercing tip (44), the needle hub being moveable from a first position wherein the distal end piercing tip (42) is within the housing to a second position wherein the distal end piercing tip (42) extends outwardly of the housing (12), a spring (23) biassing the needle hub (38) towards the first position, and a cartridge receiving member (48) mounted within the housing (12), and a plunger rod assembly (66) for exerting pressure on the cartridge receiving member (48) to move the cartridge (26) and the needle hub (38) from the first position to the second position, the plunger rod assembly (66) and the cartridge receiving member (48) being arranged such that when the cartridge rod assembly is pushed, a rotational movement is imparted to the cartridge receiving member (48) to thereby prevent access to the cartridge after injection.

### FIELD OF THE INVENTION

The present invention relates to a medical device and more particularly, relates an injection device.

### BACKGROUND OF THE INVENTION

Injection devices are utilized for administering a drug—there are many known injection devices with the most common one being a syringe. Syringes basically comprise a cylindrical tube having a needle mounted at the end, and a plunger at the other end. Intermediate the needle and the plunger is a constituent to be injected.

Syringes frequently are prefilled and used for administering many different medicaments. As is known in the art, some medicaments must be maintained at a certain temperature and this requires refrigeration of the prefilled syringe for storage purposes. This requires a substantial amount of space for storage thereof. A known problem with traditional syringes is the protection of the person having the injection. Frequently the medicament being injected can pose a risk for the personnel using the syringe. This is well known in the art and many different devices or arrangements have been proposed in order to provide protection for the person administering the injection.

Injection devices are know in the art as disclosed in U.S. Pat. No. 9,662,457 and the teachings thereof are hereby incorporated by reference.
It is an objection of the present invention to provide an injection device which does not need to be refrigerated and can accept a cartridge of the medicament to be injected.

### SUMMARY OF THE INVENTION

According to one aspect of the present invention, there is provided an injection device which comprises:
a housing, a needle hub slidable within the housing, a needle having a distal end piercing tip and proximal end piercing tip, the needle hub being movable from a first position wherein the distal piercing tip is within the housing to a second position wherein the distal end piercing tip extends outwardly of the housing, a plunger rod assembly, an interior chamber designed to receive a cartridge a containing a liquid, the cartridge having a septum and a plunger, the device being designed that the tip requires insertion of the cartridge in order to operate the same, and a cartridge holder tab which holds the cartridge by the cartridge neck to prevent operation thereof.

The injection device for the present invention will typically be utilized for administering a drug to a patient by qualified medical personnel. However, it will be understood from the scope of the invention it is not limited to such an arrangement. In some cases, the patient may self inject utilizing the device of the present invention. The device can also be utilized with animals requiring injection of a medicament.

As used herein, the distal end refers to the end from which the injection is made while the proximal end is the end closer to the hand of the person administering the injection.

The injection device may be formed of any suitable material, including glass, plastics, metallic material, combinations thereof, etc. It sufficies to say that those knowledgeable in the art will select the material most suitable for the manufacture of the device.

The housing of the injection device functions to contain the various components of the invention and conveniently may be formed of a moulded plastic or the like. The housing will also interact with the other components to assist in the motions which will be discussed in greater detail hereinbelow.

A needle hub is arranged to receive a needle or needles having a distal end piercing tip and a proximal end piercing tip. The distal end piercing tip will be a typical injection piercing tip and normally is of a relatively small diameter as is known in the art.

In the latter version of the invention, the injection device is provided with a tab such that when the cartridge is inserted in the interior chamber the needle hub will rotate to place it in a position wherein the device can be activated. Prior to this, the device cannot be activated to thereby prevent unauthorized operation of the device. This rotation of the needle hub will change its preposition such that operation of the device is allowed.

### DETAILED DESCRIPTION OF THE DRAWINGS

Having thus, generally described the invention, reference will be made to the accompanying drawings illustrating embodiments thereof, in which:

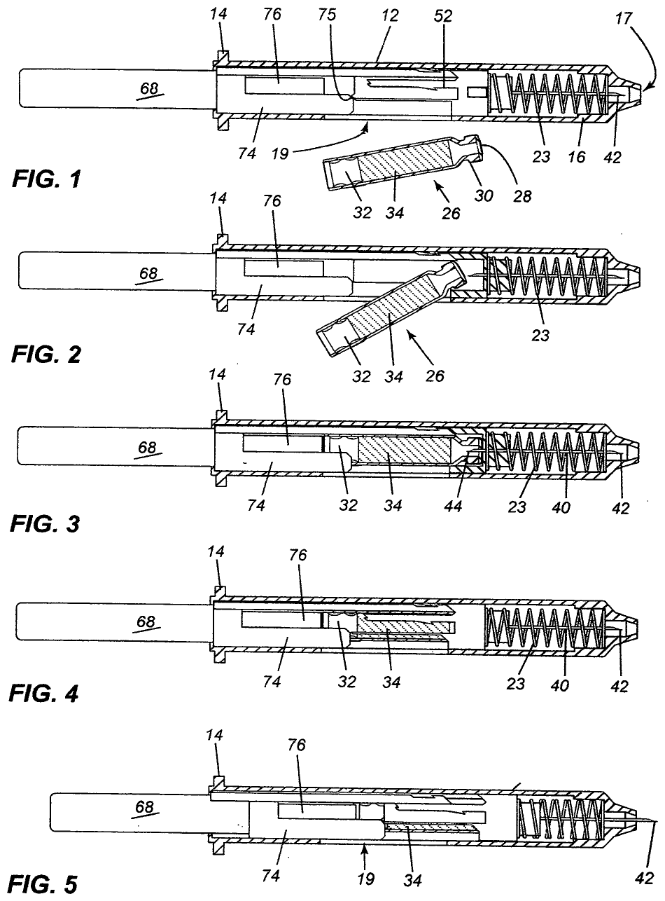

FIG. 1 is a side sectional view illustrating the device and a cartridge;

FIG. 2 is a side sectional view illustrating placement of the cartridge in the device;

FIG. 3 is a side sectional view illustrating the cartridge in the device with piercing of the septum;

FIG. 4 is a side sectional view illustrating initial movement of the cartridge within the holder;

FIG. 5 is a side sectional view illustrating further movement of the cartridge and needle hub;

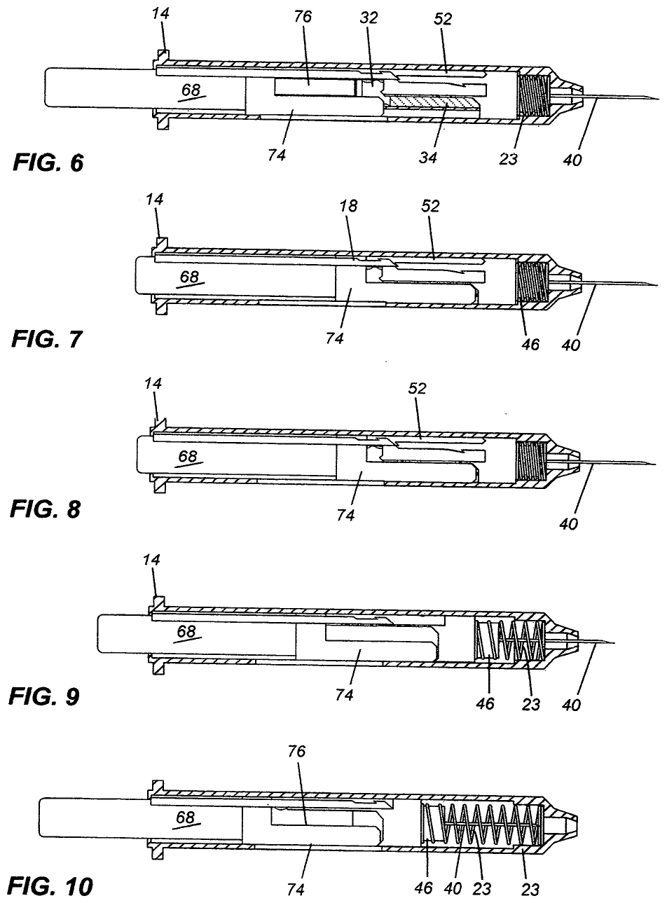

FIG. 6 is a side sectional view illustrating further movement of a plunger rod;

FIGS. 7 and 8 are side sectional views illustrating progression of the movement and injection of the cartridge contents;

FIG. 9 is a side sectional view illustrating rearward movement of the needle hub, cartridge and cartridge holder;

FIG. 10 is a side sectional view illustrating the device after injection and withdrawal of the needle;

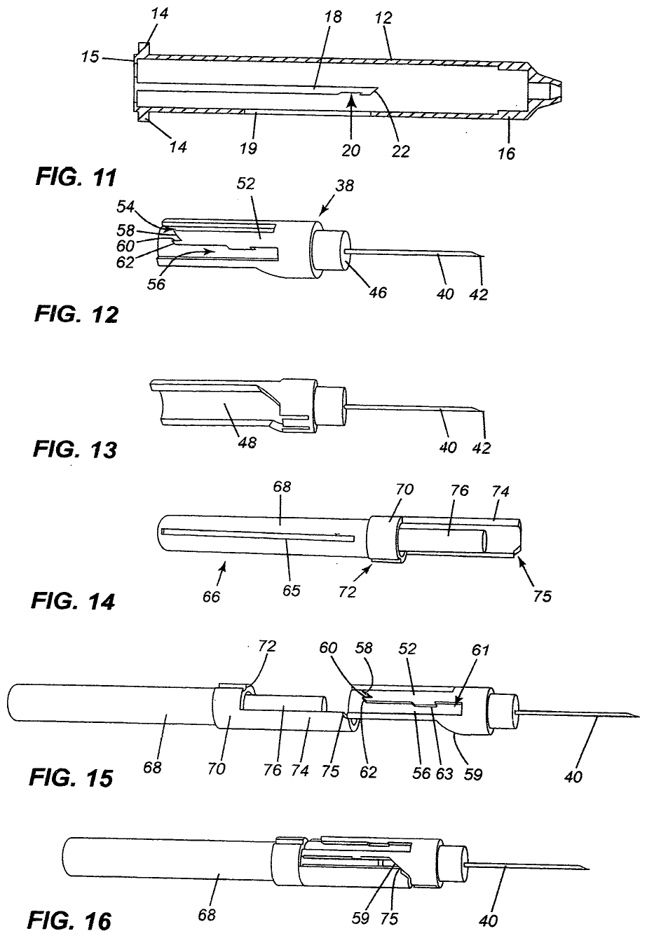

FIG. 11 is a side sectional view of the housing;

FIG. 12 is a perspective view of the needle hub and cartridge holder;

FIG. 13 is a further perspective view of the needle hub and cartridge holder;

FIG. 14 is a perspective view of the plunger rod assembly;

FIG. 15 is a perspective view illustrating the initial engagement of the plunger rod and needle hub;

FIG. 16 is a perspective view showing the position of the needle hub and plunger rod after injection; and

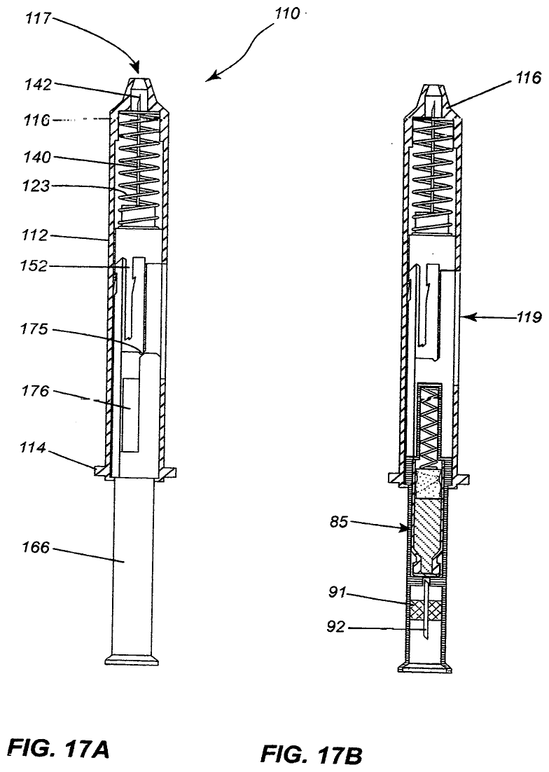

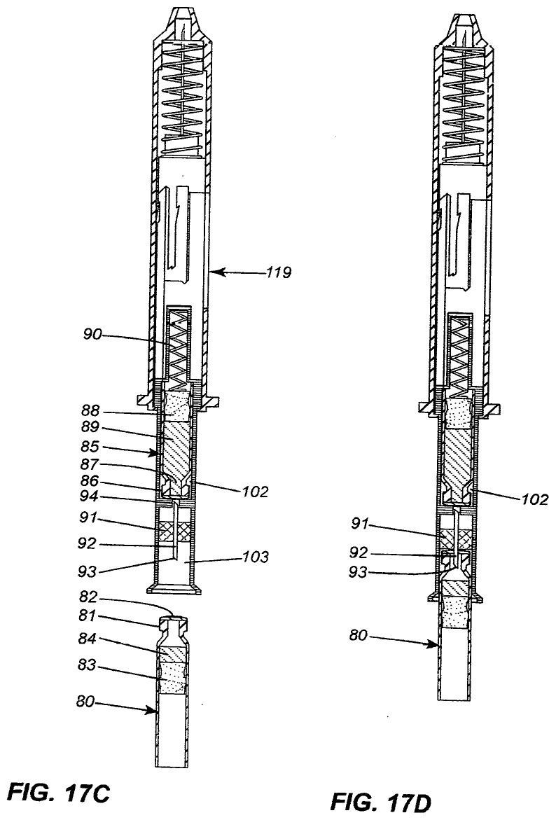

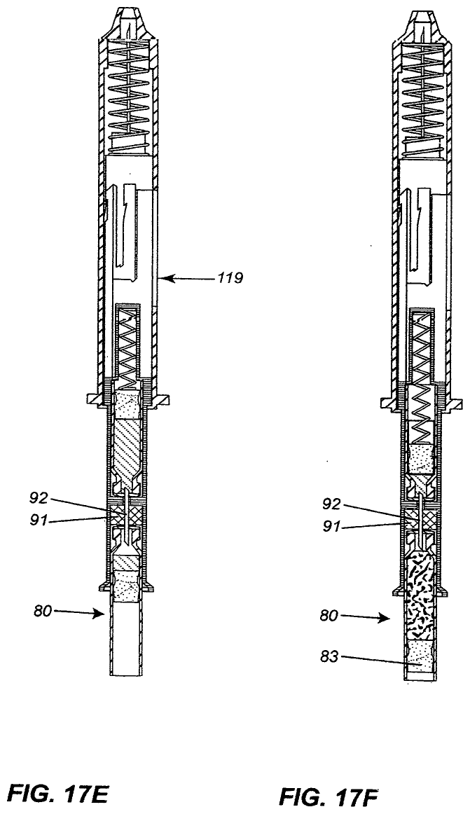

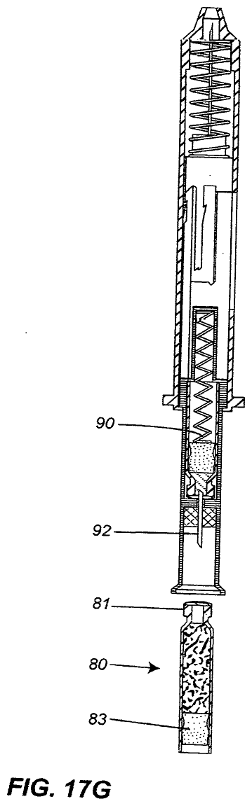

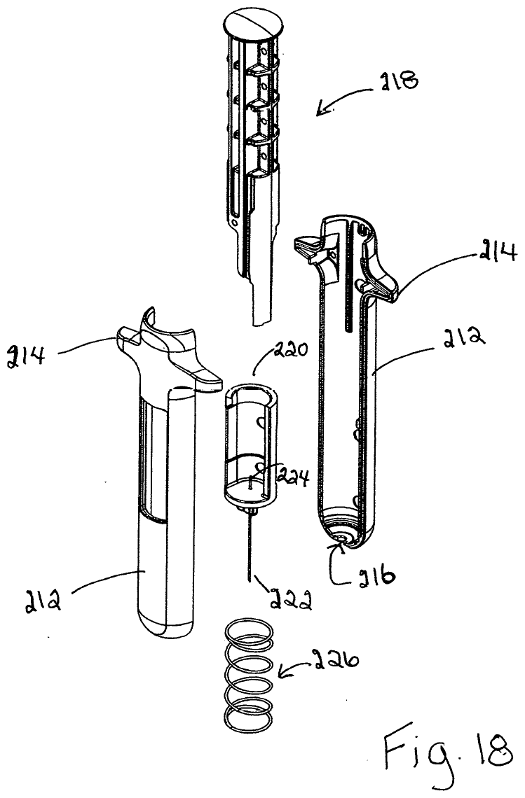

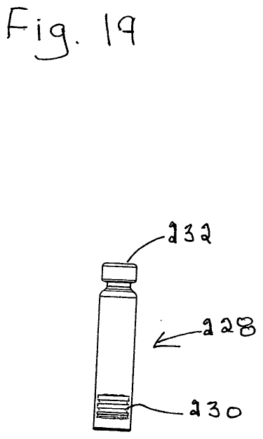

FIGS. 17A to 17G illustrate a modified version of the device.

FIG. 18 is an exploded view illustrating the various components of the injection device;

FIG. 19 is a side view of the cartridge used with the injection device;

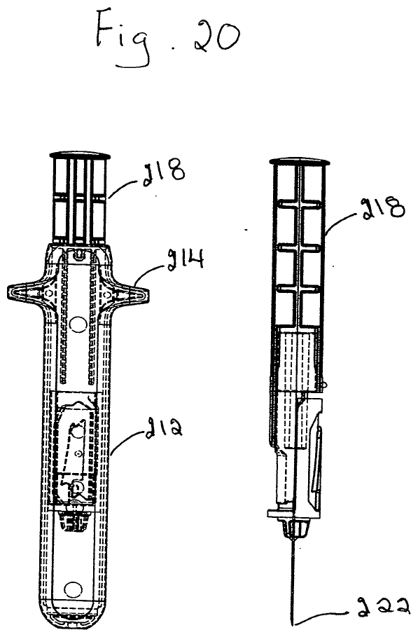

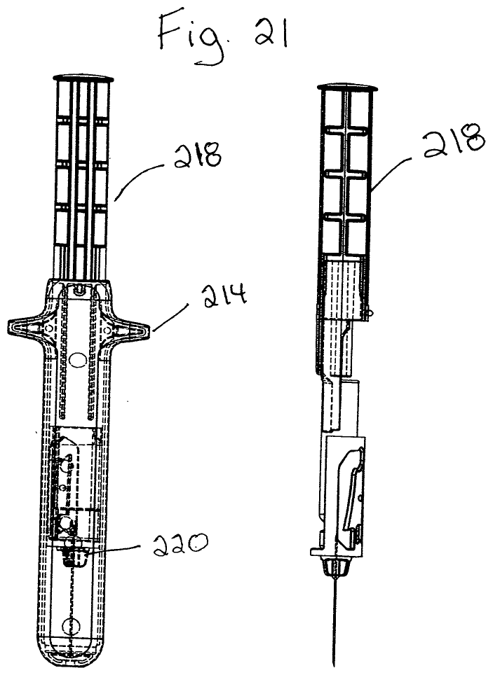

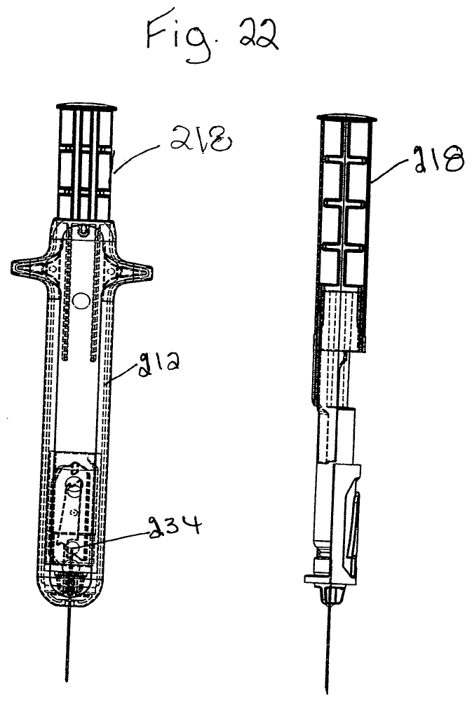

FIG. 20 is a cross sectional and cutaway views of the injection device in its initial position;

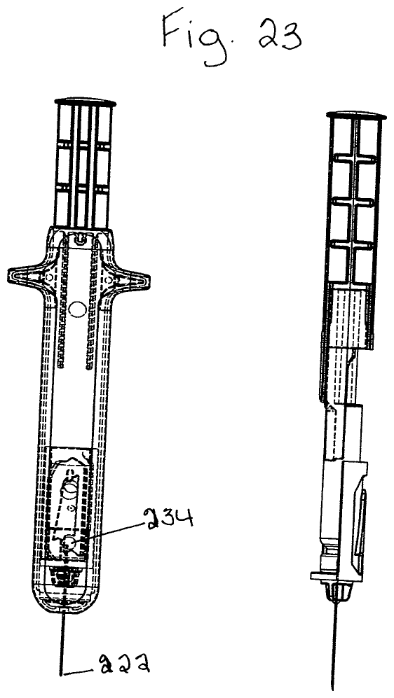

FIG. 21 is a cross-sectional and cutaway view illustrating the device when the cartridge has been inserted and the device activated;

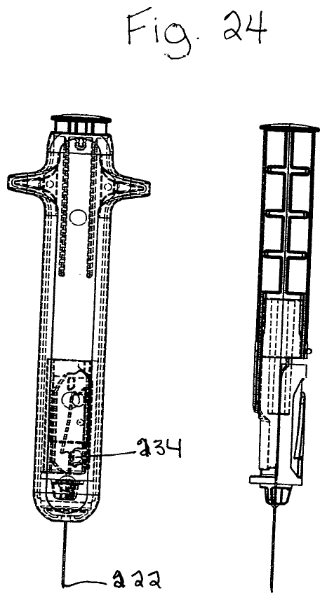

FIG. 22 illustrates cross-sectional and cutaway views of the device showing the needle enclosed;

FIG. 23 shows a cross-sectional and cutaway views when the needle is exposed and ready for injection;

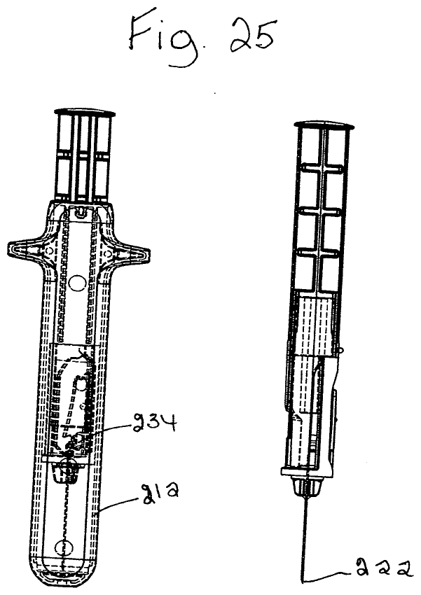

FIG. 24 shows a cross-sectional and cutaway views with the plunger being depressed and the needle ready for injection; and

FIG. 25 has a curved section and partial cutaway views of the injection device when the needle has been retracted and the device disabled.

### DETAILED DESCRIPTION OF THE INVENTION

Referring to the drawings in greater detail and by reference characters thereto, there is illustrated in FIGS. 1 to 10 the device of the present invention (generally designated by reference numeral 10) along with a cartridge generally designated by reference numeral 26.

Device 10 has an outer generally cylindrically shaped housing 12. A flange 14 is provided at the proximal end of housing 12 while at the distal end of housing 12 there is provided a cap 16. Cap 16 is screwthreadedly engaged with the distal end of housing 12. It will be noted that there is an aperture or opening 17 in cap 16 to provide for a needle passing therethrough. A slot 15 is formed in an area adjacent flange 14 for reasons discussed hereinbelow.

As may be seen in FIG. 11, there is a longitudinally extending rib 18 formed on the inner surface of housing 12. While rib 18 is shown extending a substantial length of the housing 12, it is only the upper or distal portion which is required in many embodiments. Rib 18 is provided with a recess generally designated by reference numeral 20. Rib 18 also has an angled top wall 22. An opening 19 is provided in housing 12 to permit the insertion of a cartridge as seen in FIGS. 1 to 10.

Cartridge 26 may be a standard cartridge or vial and will contain the medicament to be injected into the patient. Cartridge 26, as is conventional in the art, has a septum 28 and a cap 30 covering septum 28. A plunger 32 is mounted interiorly of the cartridge at the proximal end thereof to prevent loss of medicament 34.

A moveable needle hub 38 is mounted within housing 12 and there is provided a needle 40. Needle 40 includes a distal end piercing tip 42 for injection into the patient and a proximal piercing tip 44 which is arranged to pierce septum 28 of cartridge 26 to provide access to medicament 34.

Needle hub 38 has an upper cylindrical portion 46 and a cartridge receiving member defined by a lower semi-cylindrical wall 48 depending downwardly therefrom. On the exterior of semi-cylindrical wall 48, there is formed a recess 50 which extends partially into wall 48. A raised land portion 52 is located within recess 50 to define a first channel 54 and a second channel 56. A recess 61 is formed in second channel 56 by protrusion 63. At the lower end of raised land portion 52, there is provided a first angled bottom wall 58 which meets a side wall 60 so as to form a small notch. On the opposite side, there is provided a second angled bottom wall 62. A further angled wall section 59 is provided on needle hub 38 proximate an upper portion thereof.

A plunger rod assembly generally designated by reference numeral 66 has a lower cylindrical portion 68 at the top of which there is formed an enlarged portion 70 having a slot 72 formed therein for reasons which will be discussed hereinbelow. A rib 65 is formed on the exterior of lower cylindrical portion 68 for engagement with slot 15.

Plunger rod 66 also includes an upper partial wall 74 which extends about a central pushing rod 76 which is sized to fit within vial 26 to push plunger 32. At an upper corner of upper partial will 74, there is provided a recess 75 having a hook configuration.

Vial 26 is inserted through opening 19 in housing 12 as may be seen in FIGS. 1 and 2. If desired, it can be arranged such that vial 26 has septum 28 pierced by lower piercing end 44 upon insertion. A spring 23 extends about the upper portion of needle 40.

Initially, recess portion 75 engages end wall 62 which is tapered. The hooked nature of recess 75 ensures that the two walls remain in contact for a period of time sufficient for piercing end 44 to pierce septum 28. Continued pressure on plunger rod 66 will cause corners 75 and 71 to disengage and impart a rotational movement to needle hub 38. In turn, this will allow rib 18 to enter first channel 54. Continued pressure on plunger rod 66 will cause corner 75 to contact slanted wall 59. This causes a further rotation of needle hub 38 with respect to housing 12. This further rotation will cause the upper end of rib 18 to enter into channel 56. In so doing, pressure will be exerted until the top of rib 18 enters recess 61. The rib is then in a locked position and needle hub 38 is pushed rearwardly by spring 23.

The continued engagement of the top of rib 18 within recess 61 assures that the needle cannot be exposed as it is locked in place.

A modified device is illustrated in the embodiment of FIGS. 17A to 17G. Where components identical to those of the embodiment of FIGS. 1 to 16 are shown, similar reference numerals in the 100's are employed.

Device 110 has an overall generally cylindrically shaped housing 112. A flange 114 is situated at the proximal end of housing 112 while at the distal end of housing 112 there is provided a cap 116 which is screwthreadably engaged with the distal end of housing 112. As previously shown, there is an opening 117 in cap 116 to allow for a needle to pass therethrough.

The inner surface of housing 112 has a rib (not shown) thereon as in the previously described embodiment. Housing 112 also includes an opening 119 to permit the insertion of a cartridge.

The illustrated arrangement provides a cartridge 80 having a cap 81 and a septum 82. A plunger 83 is provided to retain a dry component 84 within cartridge 80.

A second cartridge 85 is mounted interiorly of plunger rod 166. Cartridge 85 includes a cap 86, a septum 87 and a plunger 88. Diluent 89 is mounted interiorly of cartridge 85. Plunger rod 166 has a distal interior chamber 102 and a proximal interior chamber 103.

Mounted within plunger rod 166 is a second needle hub 91 having needle 92 mounted interiorly thereof. Needle 92 includes a first piercing end 93 and a second piercing end 94. A coil spring 90 is mounted to abut plunger 88.

In operation, cartridge 80 containing the dry component is inserted into the interior of plunger rod 66. Proximal piercing end 93 of needle 92 will then puncture septum 82 of cartridge 80 as shown in FIG. 17D. Continued movement of cartridge 80 upwardly will then cause needle hub 91 to move such that distal piercing end 94 pierces septum 87 of cartridge 85 to provide a fluid passage path between the two cartridges as shown in FIG. 17E. Spring 90 will function to push plunger 88 to expel diluent 89 from cartridge 85 into cartridge 80 (FIG. 17F). Cartridge 80 may then be removed and placed interiorly of the body of housing 12 (FIG. 17G) as in the previously described embodiment. The remaining operations will remain identical to the previously described embodiment.

Referring to FIGS. 18 to 25 illustrating a different injection device generally designated by reference numeral 210 which functions in a manner somewhat similar to the previous embodiments. In this embodiment, the injection device is locked in an inoperable position prior to insertion of the cartridge therein.

Injection device 210 includes a housing 212 with a plunger rod 218 mounted therein. The injection device 210 includes a cartridge 228 which has a plunger 230 at the distal end thereof and a septum 232 at the proximal end thereof. Housing 212 is provided with finger rest 214 as is known in the art.

Interiorly of the housing 210 there is provided a needle hub 220 in which there is mounted a needle having a distal piercing tip 222 and a proximal piercing tip 224.

The injection device 210 is locked until cartridge 228 is inserted therein. In this respect, a bottom housing tooth 234 is captive in the keyway. As shown in FIG. 21, a cartridge 228 is inserted and as a result the cartridge socket moves down and causes rotation of the needle hub 220.

As illustrated in FIG. 22, the plunger rod is designed to move in an axial direction. Bottom housing fits a back stop in the keyway when the plunger rod is depressed. When the cartridge is inserted, the cartridge socket moves in a distal direction and it will generate a rotation of the cartridge socket with the cartridge back.

Following injection as shown in FIG. 25, coil spring 226 will act on needle hub 220 to cause upward movement thereof such that distal piercing tip 222 is within housing 212.

It will be understood that the above described embodiments are for purposes of illustration only and that changes and modifications may be made thereto without departing from the spirit and scope of the invention.

1. An injection device comprising:
a housing (12), an aperture (19) in a side wall of said housing (12);
a needle hub (38), a needle (40) mounted in said needle hub (38), said needle (40) having a distal end piercing tip (42) and a proximal end piercing tip (44), said needle hub (38) being slidably mounted within said housing (12), a tab member (234) mounted on said needle hub such that upon insertion of a cartridge said needle hub rotates from a position to permit subsequent activation;
said needle hub (38), being movable from a first position wherein said distal end piercing tip (42) is within said housing (12) to a second position wherein said distal end piercing tip (42) extends outwardly of said housing (12);
a spring (23) biassing said needle hub (38) towards said first position;
a cartridge receiving member (39) mounted within said housing, said cartridge receiving member (39) being accessible through said aperture (19) in said side wall of said housing (12); and
a plunger rod assembly (66) for exerting pressure on said cartridge receiving member (39) to move a cartridge (26) and said needle hub (38) from said first position to said second position, said plunger rod assembly (66) and said cartridge receiving member (39) having cooperating means such that when said plunger rod assembly is pushed to cause a cartridge septum to be pierced by said proximal end piercing tip, a rotational movement is imparted to said cartridge receiving member (39) causing said cartridge receiving member to rotate thereby positioning said cartridge receiving member (39) so as to block access to said cartridge through said aperture in said side wall of said housing.

2. The injection device of claim 1 wherein said cartridge receiving member (39) and said needle hub (38) are integral.

3. The injection device of claim 2 wherein said plunger rod assembly (66) has a pushing rod (76) and an upper partial wall (74) partially surrounding said pushing rod (76), said upper partial wall (74) having a length greater than said pushing rod (76).

4. The injection device of claim 3 wherein said upper partial wall (74) has an angled corner (75), said cartridge receiving member having a cartridge receiving member partial wall, said cartridge receiving member partial wall having a tapered corner (62) to engage with said angled corner (75) on said upper partial wall (74) to permit pushing of said cartridge receiving member (39) upwardly.

5. The injection device of claim 4 wherein contact of said upper partial wall (74) and said cartridge receiving member partial wall causes said cartridge receiving member (39) to rotate and thereby cause said cartridge receiving member partial wall to block said aperture in said side wall of said housing to thereby prevent removal of said cartridge therefrom.

6. The injection device of claim 5 wherein said housing has a protrusion on an inner surface thereof, said cartridge receiving member (39) having first and second channels, said second channel having a recess formed therein to engage said protrusion on said inner surface of said housing to prevent further movement of said cartridge receiving member (39).

7. An injection device comprising:
a housing (12), an aperture (19) in a side wall of said housing;
a needle hub (38), a needle (40) mounted in said needle hub (38), said needle (40) having a distal end piercing tip (42) and a proximal end piercing tip (44); said needle hub (38) being slidably mounted within said housing (12), said needle hub (38) being movable from a first position wherein said distal end piercing tip (42) is within said housing (12) to a second position wherein said distal end piercing tip (42) extends outwardly of said housing (12);
a spring (23) biassing said needle hub (38) towards said first position;
a cartridge receiving member (39) mounted within said housing, said cartridge receiving member (39) being accessible through said aperture (19) in said side wall of said housing (12);
a cartridge (26) mounted within said cartridge receiving member, said cartridge having a pierceable septum; and
a plunger rod assembly (66) for exerting pressure on said cartridge receiving member (39) to move said cartridge (26) and said needle hub (38) from said first position to said second position, said plunger rod assembly (66) and said cartridge receiving member (39) having cooperating means such that when said plunger rod assembly is pushed to cause said cartridge septum to be pierced by said proximal end piercing tip, a rotational movement is imparted to said cartridge receiving member (39) causing said cartridge receiving member to rotate thereby blocking access to said cartridge through said aperture in said side wall of said housing.

8. The injection device of claim 7 wherein said cartridge receiving member (39) and said needle hub (38) are integral.

9. The injection device of claim 8 wherein said plunger rod assembly (66) has a pushing rod (76) and an upper partial wall (74) partially surrounding said pushing rod (76), said upper partial wall (74) having a length greater than said pushing rod (76).

10. The injection device of claim 9 wherein said upper partial wall (74) has an angled corner (75), said cartridge receiving member (39) having a cartridge receiving member partial wall, said cartridge receiving member partial wall having a tapered corner (62) to engage with said angled corner (75) on said upper partial wall (74) to permit pushing of said cartridge receiving member (39) upwardly.

11. The injection device of claim 10 wherein contact of said upper partial wall (74) and said cartridge receiving member partial wall causes said cartridge receiving member (39) to rotate and thereby cause said cartridge receiving member partial wall to block said aperture in said side wall of said housing to thereby prevent removal of said cartridge therefrom.

12. The injection device of claim 11 wherein said housing has a protrusion on an inner surface thereof, said cartridge receiving member having first and second channels, said second channel having a recess formed therein to engage said protrusion on said inner surface of said housing to prevent further movement of said cartridge receiving member.
